(19) United States
(12) Patent Application Publication
(10) Pub. No.: US 2010/0255573 A1
(43) Pub. Date: Oct. 7, 2010

(54) EXTRACTION AND PURIFICATION OF BIOLOGIGAL CELLS USING ULTRASOUND

(76) Inventors: Damian Joseph Peter Bond, Bacup (GB); Larisa Alexandrovna Kuznetsova, Oxford (GB); Carolyn Jennifer Ruddell, Heswall (GB); Paul Birch, Liverpool (GB); William Henry Mullen, Reading (GB)

Correspondence Address: NIXON & VANDERHYE, PC, 901 NORTH GLEBE ROAD, 11TH FLOOR, ARLINGTON, VA, 22203, US

(21) Appl. No.: 12/734,671

(22) PCT Filed: Nov. 14, 2008
(86) PCT No.: PCT/GB2008/003818

§ 371 Date: May 14, 2010

(30) Foreign Application Priority Data
Nov. 14, 2007 (GB) 0722300.1
Nov. 21, 2007 (GB) 0722776.2

(51) Int. Cl. C12N 5/078

(52) U.S. Cl. 435/325

(57) ABSTRACT

A process is described in which biological cells are separated from a material sample, such as blood or soil, in which the material sample is formed as a suspension in a fluid and introduced into a chamber. One or more acoustic pressure nodes aggregate the material sample, and a flow of fluid removes soluble matter from the aggregate leaving the biological cells and other inert materials at the node(s). In one aspect of the invention the biological materials are separated from inhibitors that might render their subsequent analysis in, for example, a PCR system difficult. In another aspect of the invention a method is described to extract a clean DNA sample for such biological cells.

This invention relates to method and apparatus to separate cells and their DNA, from blood, soil samples and other materials in a form suitable for enhancement or/analysis. It is particularly useful for extracting micro-organisms, such as bacteria and viruses, and white blood cells (leucocytes) from whole blood samples and removing other materials that may interfere with diagnostic assay methods, for example, Polymerase Chain Reactions (PCR).

Extraction of low levels of cells, present in blood, food or soil samples in a form suitable for use in diagnostic assay methods such as PCR reaction systems has long been a problem. Conventional PCR processes used in identifying such biological cells do not work without cleanup steps, because factors are present alongside the target DNA that inhibit amplification of nucleic acids by PCR. Whilst the mechanisms are not totally understood, the inhibitors generally act at one or more of three aspects of the PCR process: a) in cell lysis, which is essential to release the nucleic acid from the cell, b) by degrading the released nucleic acid and preventing binding of complementary sequences and/or c) preventing efficient functioning of the polymerase enzyme. The latter is the most serious with compounds sticking to the enzyme or the nucleic acid and preventing the polymerase enzyme from replicating the DNA strands.

PCR inhibitors are widespread in body fluids or reagents used in a clinical setting (e.g. haemoglobin, urea and heparin), food constituents (e.g. organic and phenolic compounds, glycogen, fats and Ca²⁺) and environmental compounds (e.g. phenolic compounds, humic acids and heavy metals). Other more widespread inhibitors include constituents of bacterial cells, non-target DNA and contaminants, as well as laboratory items such as pollen, glove powder, laboratory plastic ware and cellulose. A fuller review of PCR inhibitors and their action is reported by Wilson, I. G. (Inhibition and facilitation of nucleic acid amplification, Applied and Environmental Microbiology, October 1997, Vol 63, No 10, p 3741-3751)

Removal of inhibitors is a critical factor in analysis by PCR and antibody binding processes. It is possible, also, that the presence of inhibitors can also have bacteriostatic effects and impact on the efficiency of bacterial cell culture using traditional techniques.

The invention provides a method to separate biological cells from a material sample comprising the steps of introducing the material sample in a fluid into a chamber, establishing at least one pressure node by means of an acoustic standing wave in the fluid, aggregating the material sample at the pressure node, releasing soluble materials from the sample either before or after the sample is introduced in a fluid into the chamber establishing a flow in the fluid in the chamber to separate the soluble material in the sample material from biological cells aggregated at the node. On completion of the separation, cells concerned may be collected or analysed in situ.

The material sample may be pre-treated to release soluble materials. A blood sample, for example, can be treated with a detergent such as saponin to burst blood cells prior its being put into the ultrasound apparatus while leaving bacteria intact. Alternatively, treatment, such as lysing, to release solubles can take place within the ultrasound apparatus itself. In a similar fashion treatment of a whole blood sample with an isotonic solution such as ammonium chloride will burst red blood cells whilst leaving leucocytes and bacteria intact. This latter is particularly useful for antibiotic activity testing or antibody labelling for testing in a method such as flow cytometry

An important outcome of the invention is that biological cells or their DNA can be extracted from a material sample in a form substantially free of inhibitors for use in subsequent analysis or amplification steps.

An acoustic standing wave field is produced by the superimposition of two waves of the same frequency travelling in opposite directions either generated from two different sources, or from one source reflected from a solid boundary. Such waves are characterized by regions of zero local pressure (acoustic pressure nodes) with spatial periodicity of half a wavelength, between which areas of maximum pressure (acoustic pressure antinodes) occur.

Ultrasound is sound with a frequency over 20,000 Hz. It has long been established that acoustic radiation force generated in an ultrasound standing wave resonator can bring evenly distributed particles/cells in aqueous suspension to the local pressure node planes. The radiation force arises because any discontinuity in the propagating phase, for example a particle, cell, droplet or bubble, acquires a position-dependent acoustic potential energy by virtue of being in the sound field. Suspended particles tend therefore to move towards and concentrate at positions of minimum acoustic potential energy. The lateral components of the radiation force, which are about two orders of magnitude smaller than the axial, act within the planes and concentrate cells/particles in a monolayer or 3-dimensional aggregates.

A particularly beneficial aspect of this invention, concerns the extraction of biological cells such as bacteria from blood samples in which a sample of blood is introduced into a acoustic chamber and the blood cells and bacteria concentrate at a pressure node formed by an acoustic standing wave, the blood cells are lysed (at the node or as a pre-treatment step), and in which fluid flow past the node separates the other blood cell contents and other soluble material in the sample from the bacteria, and on completion of separation, the bacteria are collected.

With soil, inorganic materials such as grit and sand particles range in size and generally sediment out of liquid under gravity. Smaller particles can enter the chamber and are held by the acoustic standing wave along with the target micro-organisms. The irregular shapes of the soil particles can include cavities that hold air and under the action of ultrasound these form air bubbles that oscillate and can disrupt aggregation in a flow cell. It is preferred to separate these particles by filtering the sample before introduction to the ultrasound chamber.

In a further embodiment of the invention, a number of pressure nodes are formed in the fluid, whereby separation of biological cells from the sample material occurs at a number of the nodes.

In one implementation of this embodiment, the fluid flow passes through a chamber along which a number of acoustic transducers are disposed. The biological cells are separated from the sample material at the nodes formed by acoustic standing waves at each of these transducers. The biological cells thus separated, can be passed along the chamber from one node to another by turning off the transducers in turn. This enables extremely small concentrations of cells extracted at each node to move onto to a node further along the chamber and merge with any biological cells collected at that node, thus contracting any organisms collected. In one particular such case the acoustic transducers are formed as a stack against a vertical chamber, and the chamber is filled with fluid containing the sample. Any air bubbles can be allowed to float to the top and removed before the ultra-sound transducers are turned on and the fluid flow established from the bottom. This will remove any air bubbles at the top of the chamber and prevent their disrupting aggregation. Once the ultrasound transducers are turned of any insoluble sediment present will tend to fall to the bottom of the chamber under gravity.

In practice, in a multi-transducer construct as in the previous paragraph and attaching the transducers to a single carrier layer to transmit acoustic energy into a chamber, resonance in the carrier layer decreases the effect of the system. This can be minimised by scoring the carrier layer between adjacent transducers.

In an enhancement of the invention, two or more ultrasound transducers in one or more chambers are linked in series allowing the fluid flow to pass through each transducer in turn. The first transducer or set of transducers is optimised to hold cells in multiple aggregates against a high flow rate to maximise the washing efficiency. The ultrasound is then switched off and the fluid flow passes past the last ultrasound transducer where a pressure node captures and concentrates the cells from the multiple aggregates into one single aggregate.

Whilst the biological cells and particles tend to be attracted to a main central node and form an aggregate there, conditions in the chamber can lead to multiple aggregates forming. We have found that small changes up to 1% in the ultrasound frequency (say around 1% 0.01 MHz) can be used to dislodge smaller satellite aggregates to concentrate into a larger aggregate. By using a control to change a frequency at specific time intervals, the formation of multiple aggregates into a single aggregate can be automated. Quite unexpectedly, we have found that the introduction of inert particles into fluid appears to enhance the collection of biological cells at the node. For low bacterial concentrations, the bacteria cannot be seen without magnification and the inert particles also help visualise the aggregation process and improve the efficiency of aggregate formation. Additionally they provide added resistance of the aggregate to flow, improving the separation process. We have found, surprisingly, that low numbers of biological cells, for example, as few as 5 bacteria can be separated from blood samples using this technique.

Suitable inert particles include polystyrene particles and latex particles. In the example of blood cells that have been lysed, the blood cell membranes serve the same purpose. Rather than being flushed though the system with the fluid flow, these inert particles also gather at any node. In particular inert particles of this kind are seen usefully to enhance the gathering of small bacteria and viruses at the node.

These large inert particles can be separated easily from the target micro-organisms, if desired, using conventional filtering techniques. However many applications do not require removal of the particles, which because they are inert do not interfere in subsequent reactions. Examples include PCR, ELISA, and traditional culture methods used in microbiology.

Once the biological cells have been collected (and if necessary separated from any inert materials), they can be used in convention PCR systems, free of any other organic materials that may have previously contaminated the analysis and masked any useful results. Thus the invention can be used as a preliminary step prior to a PCR analysis of a sample.

Suitable fluids for use in the invention described above are standard phosphate buffed saline (PBS) solutions, saline solutions and solutions containing growth media for the micro-organism whose presence is suspected.

In a still further embodiment of this invention, on extraction from the separation process samples containing target biological cells are passed through a dielectrophoresis chamber. In dielectrophoresis particles are polarized under the influence of an applied field and dipoles are formed that can then interact with a non-uniform electric field separating the particles on the basis of their induced charge. The charge on a cell derives from the cell membrane as well as the salts and electrolytes in the cytoplasm within the cell. While the technique produces very encouraging results in model systems, it has been less effective on real samples. The reason is because real samples tend to be high in salt, either from the sample itself (soil, blood plasma, urine, faeces) or from the culture media used to grow the bacteria. The high ionic strength of the fluids in these samples mask the charge on the cells and the dielectrophoresis can not cause a differential effect. A further aspect of the current invention is to use the ultrasound trap to form a pressure node to hold the biological cells and to change the fluid from a high ionic strength buffer to a low ionic strength buffer. Such low ionic strength buffers have around between 2 and 50 mS/m conductivity and are usually iso-osmotic. An example of a suitable low ionic strength fluid is 280 mM mannitol with an added phosphate buffered saline (PBS) solution to adjust the conductivity to the required level. Alternatively a culture medium can be used to adjust the conductivity, and this has also the advantage of providing nutrients to the micro-organisms.

The use of a dielectophoresis step provides a very effective system for purifying, then concentrating biological cells for PCR. It also is effective for the blood culture example. Blood cells which have been lysed, e.g. with Saponin which leaves the bacteria intact, can be held in the ultrasound pressure node and the contents of the blood cell separated and washed away. Blood cell membranes themselves behave as inert particles in the aggregation and separation processes as described above. In the dielectrophoresis chamber the blood cell membranes become differently charged compared to intact bacterial cells and the forces in the dielectrophoresis chamber acts differently pushing the cell membranes to one electrode and the bacterial cells to the other.

Some examples of dielectrophoresis chambers have been designed to process larger volumes in a flow through system and offer advantages with the current invention of processing larger volumes than have been practicable thus far. For example, the embodiment of the invention with vertically stacked transducers around the chamber can be designed to hold a larger volume of fluid, e.g. 10 mL, processing more biological cells than possible, for example, with current microbiology test methods. The ultrasound would present multiple aggregates, which could then be concentrated into a small volume either by a secondary ultrasound trap, or by a dielectrophoresis system so that all the biological cells in the 10 mL are concentrated into a volume suitable for PCR such as 20 μL. It is thus possible to increase the limit of detection by 500 fold based upon available bacteria numbers because of the improvements obtained as a result of optimising the PCR conditions by removing inhibitors.

An alternative means to achieve the concentration described in the previous paragraph would be to use an ultrasonic trap employing a laminar flow system as described in PCT publication WO2004/033087.

The invention can also be used with activated particles. A standard method for clean-up of nucleic acid uses particles to which free DNA will stick, such as silica or ceramic surfaces. Having washed away soluble PCR inhibitors from the sample as described above the biological cells can be lysed by a number of means. Detergent, alkali lysis, cytolytic peptides or even ultrasound can be employed. When bacterial cells are lysed in the presence of activated particles the released nucleic acid will stick. Fluid movement from acoustic streaming between node and antinode planes within the ultrasound standing wave will encourage the mixing and the likelihood of the DNA meeting the activated particles. The particles will aggregate at ultrasound pressure nodes as discussed above and a fluid flow established to wash away any remaining factors that could inhibit PCR. Hyroxyapelite or cellulose can also be used to bind bacteria (and trap them) based on charge

If the activated particles are large enough they will concentrate in the pressure nodes while the smaller particles or large bio-molecules will follow acoustic streaming in the volume. Activated particles can be ceramic or glass with proprietary surfaces such as Invitogen's ChargeSwitch. These bind DNA based on charge. Other activation treatments could be antibodies against DNA, or nucleic acid probes. The size of the activated particles will normally be no smaller than 1 μm up to 5 μm; 20 μm is typical.

A further alternative method for extracting DNA from biological cells includes the treatment of the biological cells with a thermophylic protease. This operates at a neutral pH. Thermophilic protease can either be added as a pretreatment before the sample is introduced to the acoustic chamber, or is added after the biological cells have been aggregated and washed. The aggregated cells can be heated (typically to 60° C.), either in the acoustic chamber or later, to a temperature sufficient for the thermophilic protease to digest the cell walls and holding it at that temperature for the digestion process to be completed. Further heating to a higher temperature (typically to 90° C.), deactivates the thermophilic protease. The sample is then cooled and the DNA will stick to activated particles as previously described.

Particular embodiments of the inventions are described below, by of example only, with reference to the accompanying drawings:

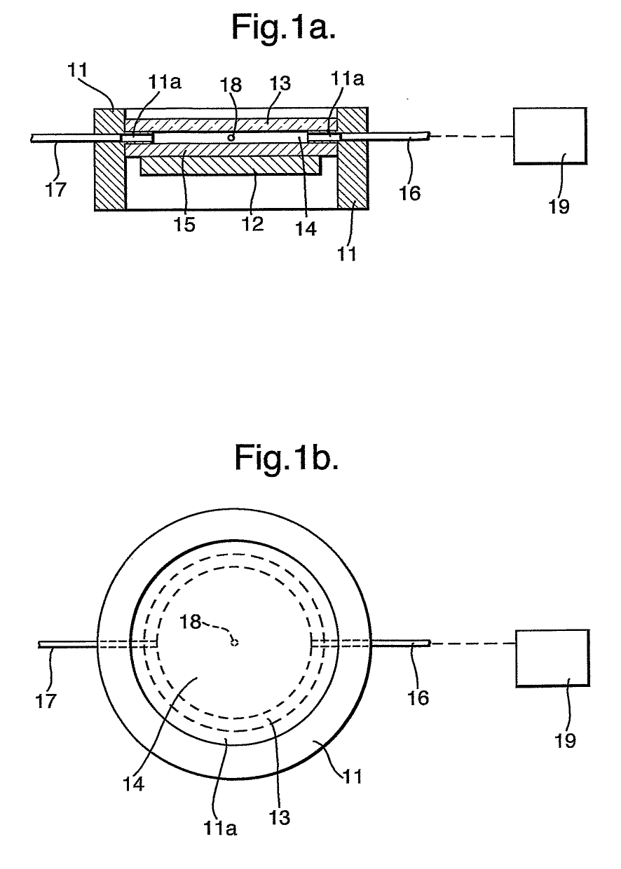

FIGS. 1a and 1b show a simple apparatus in which the micro-organism may be separated from solubles in a sample in accordance with the invention; FIG. 1a is a side view and FIG. 1b is plan view of the apparatus.

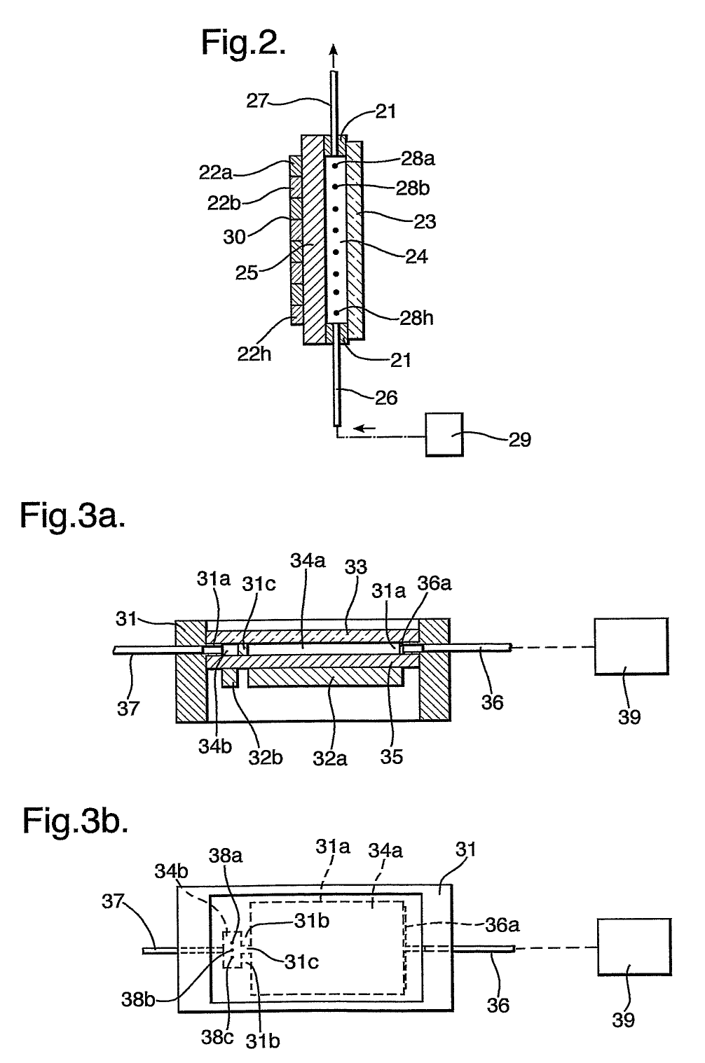

FIG. 2 illustrates the use of multiple transducers in a vertical stack in accordance with this invention;

FIGS. 3a and 3b show schematically a system comprising a large and small ultrasonic transducer used to concentrate a sample of micro-organisms; FIG. 3a is a side view and FIG. 3b is a plan view of the system;

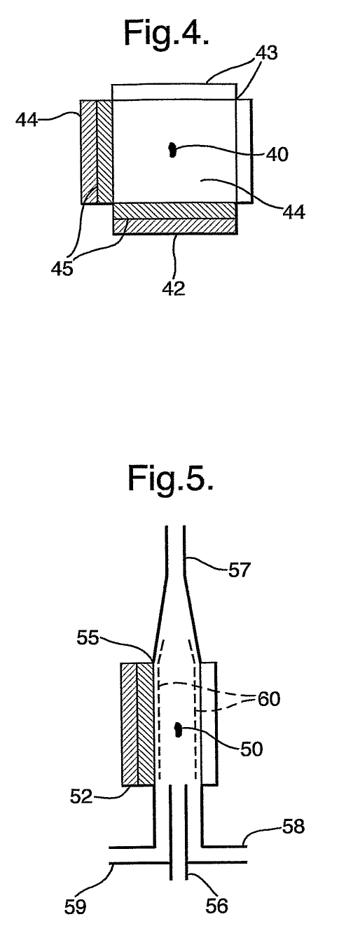

FIG. 4 is a vertical cross section of an alternative acoustic cell construct;

FIG. 5 illustrates the use of a laminar flow acoustic cell as described in PCT application publication number WO2004/033087 which can be used in connection with this invention.

In FIGS. 1a and 1b, the ultrasound device is of the kind been described in L. A. Knznetsova et al, Langmuir 2007, 23, 3009-3016.

The sample separating apparatus shown in FIGS. 1a and 1b comprises a circular stainless steel support 11, with an internal circular lip 11a on which is mounted, from below a thin stainless steel layer 15. The internal circumference of the lip and the layer 15 define the side and bottom of a chamber 14. An inlet 16 and an outlet 17 are formed through the support 11 and lip 11a to the chamber 14. A piezoelectric ultrasound transducer 12 is mounted below the layer 15 such that the centre of the transducer lies on the same axis as the centre of the chamber 14. A glass or quartz glass reflector 13 is mounted above the chamber 14; the diameter of the reflector is greater than that of the chamber 14 so that it can be sealed to the lip 11a, and seal off the chamber. The arrangement is such that the layer 15 couples the transducer 12 to the chamber 14 and the reflector 33 will allow for the creation of standing waves in any liquid in the chamber 14, when the ultrasound transducer is turned on.

The gap between the layer 15 and reflector 13 is a multiple of one half the wavelength of the intended mean frequency of the input to the ultra sound transducer. In this particular embodiment, which is suitable for use with blood samples containing bacteria operating with a transducer producing ultra sound at a frequency of 1.5 MHz, a gap of 500 μm across the chamber 14 between the layer 15 and the reflector 13 represents one ½ wavelength. When working with viruses a higher frequency would be used and consequently the gap across the chamber 14 between the layer 15 and reflector 13 would be need to be selected accordingly.

In use the chamber 14 is filled through the inlet 16 by pumping from a peristaltic pump 19 a sample suspension in a fluid comprising a standard phosphate buffer solution (PBS). In this example, the sample is blood suspected of containing bacteria. Once the chamber 14 is filled the pumping is stopped, the ultrasound transducer 12 is turned on forming one or more pressure node in the fluid. As a result blood in suspension forms aggregates 18 at pressure nodes. A medium containing the lysing agent Saponin was then pumped through the chamber 14 and out through the outlet 17. It should be noted that the efficiency of Saponin is pH dependent, and adjustments to the pH may be made to improve the efficiency of the lysing, the optimum appears to be around pH 5. The Saponin bursts the blood cells releasing their contents. Soluble materials in the sample, including the intercellular contents of the blood cells are washed away in the passing fluid and thus out of the chamber. Any micro-organisms present in the blood remains trapped at the nodes with the blood cell membranes.

Although a circular chamber 14 has been illustrated, a rectangular chamber will also work if the transducer's back electrode is etched to ensure the acoustic field's cylindrical geometry.

Selection of the precise operating parameters to use will be well within the scope of a skilled addressee. But as an illustration, a sample separating apparatus shown in FIG. 1 separated solubles from a lysed blood sample operating at a frequency of 1.45 MHz with acoustic pressure amplitude of around 1 Mpa; the initial blood cell concentration in the fluid initially pumped in was 0.3%. Once aggregates had formed at nodes, PBS containing Saponin was pumped in to establish a gentle flow velocity near an aggregate of around 1-1.5 mm/s.

In this process, secondary satellite aggregates may form and it has been found that small changes in the ultrasound frequency (eg around 0.01 MHz) can be used to dislodge such smaller satellite aggregates to concentrate into a larger aggregate. This can be achieved by using a controlled change in frequency at specific time intervals. This frequency control is easily achieved using a software programme that can control the waveform generator.

Once all solubles have left the apparatus, the micro-organisms and other inert materials remaining at nodes can be flushed out of the system by increasing the rate of flow of the PBS solution. Alternatively, micro-organisms held at nodes can be analysed, in situ.

In a development of the system shown in FIG. 1, inert particles such as polystyrene or latex particles are introduced into the sample. It has also been found that cell walls from burst red blood cells will act as inert particles for this purpose. These inert particles gather at the nodes and are not swept out of the cell with the rest of the soluble materials. It appears that use of inert particles of this kind increases the likelihood of trapping micro-organisms at the nodes, and the inert particles are particularly helpful when small numbers of bacteria or other micro-organisms are sought. In the case of very small numbers of micro-organisms, use of inert particles may be the only effective way of aggregating and washing the micro-organisms. As few as 5 bacteria have been trapped, washed and recovered using this method. The particles are also helpful in visualising the formation of the aggregate and allow the operator to adjust the frequency to speed up the rate of formation of particles into a main aggregate. This visualisation is achieved by using a video camera to look through the glass or quartz reflector 13. The overall process can also be controlled by software means, rather than by flow visualisation.

Once all the soluble materials have been flushed from the cell, a higher flow rate is established, with the ultrasound transducers remaining on, to flush out any micro-organisms and inert particles trapped at the nodes through the outlet 17. The micro-organisms and inert particles are collected. If necessary, the micro-organisms can be separated from the inert particles by conventional filtration, although their presence will not interfere with immunoassay, PCR or traditional microbiology culture techniques. The micro-organisms, now substantially free of any soluble materials or inhibitors from the sample can be used in conventional PCR apparatus or for other forms of analysis.

A similar approach is adopted for soil samples. In this case too, inert inorganic particles such as grit and sand may gather at the pressure nodes with the sought for micro-organisms, whereas organic materials, particularly nucleic acids, humic acids or other PCR inhibitors present in the soil are flushed clear out by the fluid flow. Once again inert particles such as polystyrene and latex have been found to enhance the robustness of the process for trapping small numbers of biological cells at the nodes. Once soluble material has been washed from the cell, the biological cells trapped at nodes are flushed though and any organisms are analysed.

Although apparatus FIG. 1 has been described in relation to the separation of micro organisms, a similar technique can be used for separation of white blood cells from a whole blood sample. In this case lysing would be carried out using an isotonic solution such as ammonium chloride to burst of red blood cells while leaving leucocytes and any bacteria intact.

In FIG. 2, a rectangular stainless steel support 21 supports a layer of stainless steel 25. A chamber 24 in the form of a rectangular cross sectioned duct is formed between the layer 25 and the support 21. The chamber 24 has an inlet 26 and outlet 27 as before. A series of ultrasound transducers 22a to 22h are disposed along the side of the duct, with the layer 25 coupling the transducers to the chamber. A glass or quartz glass reflector 23 is sealed to the support and closes off the chamber. The gap between the layer 25 and the reflector 23 across the chamber 24 is again a multiple of one half the wavelengths of the mean designed ultrasound frequency of operation. Typical operational frequencies and gaps are as described with reference to FIGS. 1a and 1b.

The stainless steel layer 25 has a score or cut 30 between each transducer 22. This appears to minimises the combined effect of vibrating transducers resonating the stainless steel layer 25 (the carrier layer); however, whatever the mechanism, the scoring 30 improves the overall effectiveness of the system.

A peristaltic pump 29 is used to charge the chamber 24 with the sample, e.g. blood sample dispersed in a buffer fluid. The ultrasound transducers 22a to 22h are turned on, with the result that nodes 28a, 28b, . . . 28h are formed in the fluid within the chamber 24 between the layer 25 and the reflector 23. Cells will gather at the nodes. This arrangement is designed with the intention of forming multiple pressure nodes and therefore aggregates of cells. Once aggregates are formed, a flow of PBS fluid, containing the detergent Saponin to lyse the blood cells, is introduced through inlet 26 by use of the peristaltic pump 29. Soluble materials including intracellular blood products are separated from any biological cells present in the sample and are flushed out, leaving biological cells and cell membranes at the nodes. By turning off the highest ultrasound transducer 22a any biological cells held at nodes 28a will drop to the next nodes 28b enriching the numbers of cells held there. The process is then repeated by turning off ultrasound transducer 22b. This is done for each transducer in the stack until all the biological cells are concentrated in the nodes 28h formed by ultrasound transducer 22h. As before the separated biological cells can be collected by flushing through at a higher flow rate once all soluble materials have left the system.

Inert particles can be used in this system in the same way as described previously in respect of FIG. 1. Similarly if soil samples are used, inset inorganic particles may be gathered along with the target biological cells and may need to be filtered. Best results appear to be achieved with the inlet 26 at the bottom of the chamber and the outlet 27 at the top. This may be because air-bubbles in the fluid rise to the top of the chamber and are removed by the fluid flow without interfering with the aggregation and washing process. Large inert particles drop to the bottom of the chamber.

In FIGS. 3a and 3b, a rectangular stainless steel support 31, frames a central rectangular aperture extending for about three quarters of the length of the support, and a smaller rectangular aperture towards one end. A lip 31a extends around the two apertures with a pair of opposed fingers 31b extending from each long side of the support to define a narrow channel 31c between the large and smaller apertures. A stainless steel layer 35 is mounted on the lip 31a below the apertures. Two rectangular chambers 34a and 34b, one 34a being much the larger, are thus formed between the lip 31a, the fingers 31b and stainless steel layer 35 and joined by the narrow channel 31c.

As before, a glass or glass quartz reflector 33 closes off the two chambers on is sealed to the lip 31a of the support 31. Transducers 32a and 32b are mounted below the layer 35. A larger transducer 32a is positioned to act upon chamber 34a and a smaller transducer 32b, to act on the smaller chamber 32b. The layer 35 thus couples the transducers 32a and 32b to the chambers 34a and 34b respectively. A cut in the stainless steel layer 35 on the surface below the apertures and in between the transducers 32a and 32b will minimise vibrations from one transducer affecting the adjacent chamber

In all the examples the transducers can be notched or their back electrodes can be etched so that they will cause multiple nodes. However, it is particularly advantageous that the smaller transducer's 32b back electrode is etched so that several nodes are formed across the flow in any fluid in the chamber 34b when transducer 32b is operated. Alternatively transducer 32b can be notched to achieve the same effect.

Inlets 36 to the larger chamber 34a and outlet 37 from the smaller chamber 34b are formed in the stainless steel 31. With rectangular chambers, consistency of any fluid flow across the width of the chamber is assisted by the inlet to the chamber itself being formed as a transverse slot 36a across the side of the chamber 34a. On the outlet side, the outlet 37 is gained by a small orifice 37a in the side of chamber 34b.

In use, the ultrasound transducer 32a is turned on and chamber 34a charged by a peristaltic pump 39 through inlet 36 with a fluid containing a sample in a PBS solution. PBS fluid alone is now pumped in and any solubles will now be washed from the aggregates through channel 31c and out of the apparatus via the orifice 37a and outlet 37. The slot 36a distributes the inward flow of fluid evenly across the width of chamber 34a and minimises the risk of too high a flow at any point within chamber 34a which might breaking up the aggregates.

Once it is established that there are no further solubles in the fluid leaving the outlet, transducer 32b is turned on and transducer 32a turned off. A gentle fluid flow is maintained through the channel 31c. This has the effect of moving any biological cells and inert solids gathered in the larger chamber 34a into the smaller chamber 34b. These materials again form aggregates 38a, 38b, 38c etc at the nodes in chamber 34b whose small size of chamber 34b results in their being concentrated in a small volume. The benefit of etching transducer 32b's back electrode is seen in this context. Without etching it is possible that only one node will form in chamber 34b and cells may go around it with the risk of becoming lost. With multiple nodes, that risk is significantly reduced.

As before, once the biological cells have been gathered in chamber 34b they may be analysed in situ, or the flow rate increased to flush them out for collection and further treatment in, for example, in a dielectrophoresis chamber, passed directly to a PCR apparatus.

In FIG. 4, apparatus is shown in which greater acoustic power can be brought to bear on a sample. This particular arrangement is useful when the sample size is likely to be large (e.g. 1 mL) and there is a risk that the washing process in a smaller chamber will take too long a time for it to be useful. The apparatus comprises a square cross-section chamber 44 having orthogonally mounted ultrasound transducers 42 attached to thin stainless coupling layers 45 forming two walls of the chamber 44. Opposite each f the transducers 42, reflectors 43 form the other two walls of the chamber. The chamber is 10 mm across. With both transducers operating a strong acoustic signals overlap and form multiple nodes where cells aggregate. The strong acoustic power concentrated at these nodes will hold any cells in the sample 40 strongly in place. Although the figure shows one node, in practice there will be many—hundreds form in practice. This is an effective approach to wash inhibitors away in a large sample that contains low numbers of biological cells. As with other chamber designs, inclusion of inert particles can help improve aggregation and retention of small numbers of bacterial cells.

A further alternative acoustic chamber construction to be used with the present invention is shown in FIG. 5. The acoustic chamber 54 comprises a stainless steel layer 55 opposite a reflector 53. An acoustic transducer 52 is coupled to the stainless steel layer 55. The chamber is provided with an inlet 56 and outlet 57. Additional inlets 58 and 59 are placed each side of inlet 56. When a sample is introduced through inlet 56, a neutral buffer solution is also introduced through inlet 58 and 59 to establish a laminar flow 60 either side of the sample 50. The cells in the sample are therefore directed into the node of the ultrasound standing wave, avoiding the need for lateral forces to slowly push them into aggregates and the subsequent frequency modulation to form a single aggregate as with other chamber designs. Actuation of the transducer 52 traps the sample 50 into a very small volume at the centre of the chamber 54. In one example, a chamber based around a 1.5 MHz transducer had 3 incoming channels of width 1.5 mm leading into a main chamber of width 4 mm and an exit aperture of 2 mm. The active area of the transducer was etched as a circle of 4 mm diameter in the main chamber. 200 μL of 2.81 uM polystyrene particles at 0.2% w/v concentration were flowed through the central incoming channel over a 10 minute period whilst a frequency of 1.681 MHz was applied to the transducer (25V at the transducer). The polystyrene particles formed into an loose aggregate. After 10 minutes, the frequency was switched to 1.675 MHz (25 V at the transducer) to form a centralised uniform aggregate. The liquid in the central incoming channel was changed to bacterial suspension at a concentration of 6×10³ bacteria/mL and 1 mL was flowed through over 50 minutes. The chamber contents were removed and plated out to obtain bacterial counts. The results confirmed that 60% of the bacteria in the initial 1 mL suspension had been retained in the aggregate.

The experiment was repeated as described in the paragraph above, using bacteria in a blood culture simulation (using whole blood at 20% dilution of normal haematocrit). The blood cells were lysed and 60% recovery of bacteria from the original 1 mL were recorded.

A chamber of this kind could replace the second chamber 34b in FIG. 3 to ensure a highly concentrated sample, or indeed it may be used to replace both chambers in that figure.

Variations are possible. Where the original sample contained blood, the fluid pumped in to wash away soluble materials could additionally contain a suitable lysing agent to cells collected in the aggregates. In FIG. 3, rectangular chambers 34a and 34b are shown, these could be circular instead, or there could be one of each. Parameters such as typical frequency, amplitudes are selected to suit the materials and biological cells concerned, but for blood samples would typically be similar to those indicated for FIGS. 1.

Separation of viruses would require a higher frequency and pro-rata a smaller gap between the layers 15,35,35,45 and the reflectors.

The material sample can be pre-treated to release solubles contained in it, prior to its introduction into the chambers. In the case of blood, for example, it can be mixed with a lysing agent to burst the cells prior to its being pumped into the apparatus instead of being lysed within the chambers themselves.

With each of the illustrated examples a dielectrophoresis chamber can be used as a further stage of the process. Once the desired biological cells have been gathered in aggregates at a node(s), the fluid passing though the system is changed to a low ionic buffer solution. Once the original high ionic buffer solution has completely left the chamber the relevant transducer can be turned off and the biological cells together with any inert material gathered passed into a dielectrophoresis chamber. This chamber will enable cells to be sorted based upon their charge. The charge on a cell derives from the cell membrane as well as the salts and electrolytes in the cytoplasm within the cell.

The problem of the known dielectorphoresis cells is overcome since salt, either from the sample itself (soil, blood plasma, urine, faeces) or from the culture media used to grow the bacteria has been flushed away during the ultrasound separation process described. The detrimental impact of the previous high ionic content of samples used in dielectorphoresis cells is avoided and better results obtained. The charge on the blood cell membranes is different to the charge on the intact bacterial cells and the forces in the dielectrophoresis chamber acts differently pushing the cell membranes to one electrode and the bacterial cells to the other, thus enabling their separation.

In each of the examples, activated particles to adsorb nucleic acids can be used. There are a number of known particles such as silica or ceramics to which free DNA will adhere. Such particles can be introduced into the chamber in which the nodes are formed.

When bacteria are being sought, once the soluble inhibitors from the sample have been removed as described above, the bacterial cells can be lysed by a number of means, detergents, alkali lysis, cytolytic peptides or even ultrasound can be used. When bacterial cells are lysed in the presence of activated particles the released nucleic acid will stick to the activated particles. Fluid movement from acoustic streaming between the pressure nodes within the ultrasound standing wave will encourage the mixing and the likelihood of the DNA meeting the activated particle. The particles will aggregate into an ultrasound pressure node as discussed above and a fluid flow established to wash away any remaining factors, such as the nucleic acids, that could inhibit PCR.

Particles suitable for cell binding can be introduced, and these will further improve the probability of biological cells being captured. These particles may be a metal hydroxide such as titanous hydroxide, zirconium hydroxide, hafnium hydroxide or hydroxyapatite, or particles coated with such hydroxides which are know to bind most species of bacteria. These binding particles can usefully have para-magnetic cores, which will allow the particles together with the bound cells to be trapped when they are flushed out of the acoustic apparatus.

Where sample materials, such as earth, contain air and/or large grit particles, it would be appropriate to filter the suspension of the material in the selected buffer fluid before it is introduced into the chamber.

A further possibility is the use of inert particles to help adjust the ultrasound frequency. By introducing inert particles or placing inert particles into the chamber(s), the aggregate formation can be observed and the ultrasound frequency can varied. This may be particularly useful when using apparatus designed to separate one biological cell from another, or when some degree of fine tuning is needed. An example of this latter situation is where a whole blood sample is being analysed and it is desired to separate bacteria from the white blood cells.

A further possibility includes the use of thermophilic protease. When biological cells are mixed with a protease of this kind and heated to 60° C. for 10 minutes, the protease is active and digests the bacterial cell walls. Any RNAse present is inactivated and digested as well. When heated further to 90° C. the protease is denatured. Any inhibitors present will also be digested or deactivated. This process can be carried out in the acoustic chambers described or subsequently. The released DNA can be bound to a nucleic acid binding material, such a silica particles, as the acoustic chamber cools. If necessary, further washing can take place. If the nucleic acid binding material has a para-magnetic core, these particles together with the bound DNA can be trapped using a permanent magnet. If this process is done within an acoustic cell the magnet can be mounted at the outlet. In a two chamber system, in which the second acoustic chamber is used to concentrate the sample, any additional washing can be achieved by passing the sample back to the first chamber.

1.-69. (canceled)

70. A method of separating biological cells and/or their contents contained in a sample from the remainder of the sample wherein the improvement comprises the steps of:
introducing the sample material in a fluid into a chamber;
establishing at least one pressure node in the chamber by means of an acoustic standing wave in the fluid;
aggregating the sample material at the node establishing a flow in the fluid to separate the soluble matter in the sample material from cells aggregated at the node;
mixing the sample material with a biological binding material; and
allowing the biological binding material to bind to biological material in the sample;
said mixing of the sample material with the biological binder taking place before or after the sample is introduced into the chamber.

71. A method according to claim 70 wherein the improvement comprises treating the sample is treated to release soluble materials in biological cells in the sample either before or after the sample is introduced in a fluid into the chamber; and
thereby releasing soluble materials in biological cells in the sample.

72. A method according to claim 71 wherein the improvement comprises analysing biological cells after completion of the separation of soluble matter from the sample material.

73. A method according to claim 70 wherein the improvement comprises:
using a low ionic fluid in the flow to replace the fluid in which the cells were originally;
passing collected biological cells in a suspension into a dielectrophoresis chamber;
separating in the dielectrophoresis chamber biological cells from any other material, such as cells walls, in the suspension.

74. A method according to claim 71 wherein
the material sample is blood;
the blood cells are lysed to release their contents; and
separating the soluble material content of the blood cells from particles or intact biological cells by flow of the fluid.

75. A method according to claim 71 wherein the biological cells are leucocytes; and the method includes releasing soluble materials from the sample using an isotonic solution such as ammonium chloride.

76. A method according to claim 75 wherein the method is part of an antibody labelling methodology.

77. A method according to claim 74 wherein the improvement includes the step of releasing DNA from separated biological cells by lysing.

78. A method according to claim 70 wherein the improvement comprises the step of introducing activated particles comprising charged surfaces, or antibody or ligand coatings suitable for binding haptens, proteins, cells or nucleic acid into the chamber.

79. A method according to claim 78 wherein the activated particles are paramagnetic.

80. A method according to claim 79 wherein the activated particles comprise ceramic coated on a paramagnetic core or silica coated on a para-magnetic core.

81. A method according to claim 79 a primary capture mechanism for a antigen or molecule and that the fluid flow can introduce a secondary binding event for detection, such as an immunoassay mechanism.

82. A method according to claims 77 wherein the improvement comprises the step of adsorbing released DNA onto activated particles comprising nucleic acid binding material on a paramagnetic core.

83. A method according to claim 79 wherein the improvement comprises the step of collecting the activated particles on at least one magnet.

84. A method according to claim 70 wherein the biological binding materials are particles selected from the group comprising metal hydroxide selected from the group comprising titanous hydroxide, zirconium hydroxide, hafnium hydroxide or hydroxyapatite, another metal hydroxide, cellulose, or particles coated with metal hydroxide.

85. A method according to claim 70 wherein the improvement comprises the steps of:
forming one or more nodes is formed in a chamber in the region of one transducer;
trapping biological cells in said nodes;
turning that transducer off;
releasing said trapped biological cells;
forming further nodes in the region of a second transducer; and
aggregating said released biological cells at the node(s) created by the second transducer.

86. A method of separating DNA in biological cells in a sample from the remainder of the sample wherein the improvement comprises the steps of:
introducing the sample material in a fluid into a chamber;
establishing at least one pressure node in the chamber by means of an acoustic standing wave in the fluid;
aggregating the sample material at the node establishing a flow in the fluid to separate the soluble matter in the sample material from cells aggregated at the node;
mixing the sample material with a biological binding material; and
allowing the biological binding material to bind to biological material in the sample, said mixing of the sample material with the biological binder taking place before or after the sample is introduced into the chamber;
treating the sample is treated to release soluble materials in biological cells in the sample either before or after the sample is introduced in a fluid into the chamber, thereby releasing soluble materials in biological cells in the sample;
introducing activated particles comprising nucleic acid binding material into the chamber; and
adsorbing released DNA onto the activated particles comprising nucleic acid binding material.

87. A method according to claim 86 wherein the DNA is released from speared cell by lysing

88. A method according to claim 86 wherein activated particle comprising nucleic acid binding material have a para-magnetic core.

89. A method according to claim 88 wherein the improvement comprises collecting the said particles on a magnet.
